(19) United States
(12) Patent Application Publication
(10) Pub. No.: US 2012/0231098 A1
(43) Pub. Date: Sep. 13, 2012

(54) Weight loss composition

(76) Inventors: Stephen J. Turano, Clearwater, FL (US); Ronald Hayter, Clearwater, FL (US)

(21) Appl. No.: 13/385,864

(22) Filed: Mar. 9, 2012

Related U.S. Application Data
(60) Provisional application No. 61/451,908, filed on Mar. 11, 2011.

(51) Int. Cl. A61K 36/76; A61P 29/00; A61P 3/04

(52) U.S. Cl. 424/769

(57) ABSTRACT

The present invention relates to herbal and chemical compositions for inducing/improving weight loss. The herbal components of the composition can include, among others, white willow bark extract. The chemical components of the composition can include, among others, caffeine and phosphatidylserine.

### CROSS-REFERENCES TO RELATED APPLICATIONS

This non-provisional application depends on and is a continuation of that certain provisional application number 61/451,908 filed on 11 Mar. 2011.

### STATEMENT REGARDING FEDERALLY SPONSORED RESEARCH OR DEVELOPMENT

Not applicable.

### MICROFICHE APPENDIX

Not applicable.

### FIELD OF INVENTION

This invention relates to herbal supplements. Specifically, the invention provides a composition and method to promote weight loss.

### SUMMARY OF INVENTION

Obesity is a growing epidemic in the United States that is in part caused by diet and lack of exercise. Diets having a high fat content as well as the repeated ingestion of refined foods and sugars coupled with low fiber and vegetable intake contribute to the obesity epidemic. Diet, lack of exercise and the natural aging process causes deterioration in the manner in which the body metabolizes blood glucose. When the body cannot properly metabolize blood glucose, there is a tendency to store glucose as fat. There is a connection between insulin resistance and increased visceral adiposity, thus when glucose regulation is unbalanced, a greater propensity for adiposity exists. There is also a link between obesity and several diseases such as, heart disease, hypertension, Type II diabetes, and insulin resistance.

The present invention provides a nutritional weight loss composition that increases metabolism, decreases hunger, increases fat utilization, and reduces cortisol levels in the body.

The weight loss composition of the present invention is a composition comprising at least one thermogenic ingredient, at least one anti-inflammatory ingredient, and at least one cortisol level reducing ingredient.

The thermogenic ingredient can be caffeine. Further, the form of the caffeine can be caffeine anhydrous. The anti-inflammatory ingredient can be white willow bark extract. The ingredient that reduces cortisol levels can be phosphatidylserine.

The amount of caffeine in the weight loss composition can be between about 1 mg and about 1200 mg. A beneficial amount of caffeine in the composition is about 200 mg per dose. The amount of white willow bark extract can be between about 1 mg and about 1650 mg. A beneficial amount of white willow bark extract in the composition is about 550 mg per dose. The amount of phosphatidylserine can be between about 1 mg and about 1200 mg. A beneficial amount of phosphatidylserine in the composition is about 200 mg per dose.

A method of promoting weight loss in a subject is also presented in which the subject is administered an effective amount of a composition comprising caffeine, white willow bark extract, and phosphatidylserine. The weight loss composition can be administered orally such as in a tablet or capsule. In this form, the dosage can be 1 capsule administered about 3 times per day for 5 consecutive days about 30 minutes prior to each meal. Each capsule can be taken with about 8 oz. of water.

### DETAILED DESCRIPTION OF A PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range, to the tenth of the unit. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

The term “about” as used herein is not intended to limit the scope of the invention but instead encompass the specified material, parameter or step as well as those that do not materially affect the basic and novel characteristics of the invention.

The term “composition” as used herein describes an agent, compound, chemical, or extract that can be administered or tested by the present invention as promoting weight loss in a subject. The chemical can be of any composition such as inorganic, organic, or a biomolecule. A biomolecule can be a molecule of any biological origin that can be found in or produced by, at least in part, a cell. This definition includes, but is not limited to, polypeptides, lipids, nucleic acids, carbohydrates and combinations thereof. “Composition” is used interchangeably herein with “compound”, “agent”, “chemical”, “drug”, “ingredient” and “extract”.

“Subject” is used to describe an animal, preferably a human, to whom treatment is administered, including prophylactic treatment with the compositions of the present invention. “Subject” and “patient” are used interchangeably herein.

A “effective amount” as used herein is defined as concentrations or amounts of components which are sufficient to effect beneficial or desired clinical results, including, but not limited to, promoting weight loss; increasing metabolism; decreasing hunger; increasing fat utilization; reducing cortisol levels in the body; increasing feelings of satiety; and increasing thermogenesis. Compositions of the present invention can be used to effect a favorable change in the condition whether that change is an improvement or a complete elimination of symptoms due to obesity. In accordance with the present invention, a suitable single dose size is a dose that is capable of preventing or alleviating (reducing or eliminating) a symptom in a subject when administered one or more times over a suitable time period. One of skill in the art can readily determine appropriate single dose sizes for systemic administration based on the size of the animal and the route of administration. The effective amount of the compositions of the present invention encompasses providing weight loss treatment or enhancing weight loss treatment without causing significant side effects or adverse reactions.

The term “administrating” or “administration” as used herein are defined as the process by which the compositions of the present invention are delivered to the individual for treatment or prevention purposes. The composition can be delivered orally, rectally, percutaneously, or by injection in dosage unit formulations containing conventional nontoxic pharmaceutically acceptable carriers, adjuvants, and vehicles as desired. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For injection compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g. as a transdermal patch, or a spot-on treatment.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin EW [1995] Easton, Pa., Mack Publishing Company, 19^{th} ed.) describes formulations which can be used in connection with the subject invention.

Changes in metabolic rate can be affected by factors such as ambient temperature, body size/mass, the proportion of muscle tissue versus fat in body weight, level of physical exertion, and genetic predisposition for a higher or lower resting metabolic rate. In addition, pharmacological, nutritional, and physiological factors can also affect metabolic rate. (Bray G. A., Nutrition. October 2000. 16(10):953-60) Increases in metabolic rate are associated with increased thermogenesis which in turn is associated with increased weight loss since more calories are being burned by the body.

In general, drugs used to treat obesity can be divided into three main groups: those that reduce food intake; those that alter metabolism; and those that increase thermogenesis. The composition of the present invention is designed to address all three main groups of drugs used to treat obesity.

The composition of the present invention comprises as active ingredients, at least one thermogenic, at least one anti-inflammatory, and at least one cortisol level reducing ingredient. These active ingredients can be administered in a pharmacologically acceptable carrier along with certain inactive ingredients.

### Thermogenic Compounds

Thermogenic compounds are those compounds which boost the body's metabolism and assist in burning fat stores as energy. Thermogenic compounds increase the basal metabolic rate (BMR) of the body to burn more calories overall.

The thermogenic ingredient can be any compound that is capable of inducing a thermogenic effect when administered to a subject. Examples of thermogenic ingredients are: caffeine, including caffeine anhydrous; catechins, including EGCG found in green tea; polyphenols; bitter orange; capsaicin; ginger; guar gum; pyruvate; leptin; chitosan; pepper; compounds containing ephedrine and pseudoephedrine; compounds containing synephrine; and compounds, mixtures or extracts thereof.

### Caffeine

Caffeine is a naturally occurring xanthine alkaloid found in some plants. It is a common additive to food products for human consumption and as such, exhibits many beneficial effects. It is commonly used as a supplement to the central nervous system where it acts as a neuro-stimulant and performance enhancer.

Biochemically, caffeine binds to, but does not activate, adenosine receptors. Adenosine receptors are normally activated by adenosine to induce sleep, thus caffeine acts as a stimulant by occupying the receptor without activating it. By antagonizing adenosine receptors, caffeine increases levels of intracellular cAMP, which is an important signaling molecule in thermogenesis. (Ramkumar V. et al., Multiple components of the A1 adenosine receptor-adenylate cyclase system are regulated in rat cerebral cortex by chronic caffeine ingestion. J Clin Invest. 1988. 82(1): 242-7)

Caffeine also enables the increase of cAMP levels through the inhibition of phosphodiesterases which specifically degrade cAMP. The actions of caffeine with regard to the cAMP system lead to an increase in the release of epinephrine and norepinephrine. Epinephrine and norepinephrine use cAMP for signaling thus increased levels of cAMP results in increased adrenergic signaling which in turn induces lipolysis during which thermogenesis can expend energy in turn leading to body fat loss. Caffeine dilates coronary arteries and bronchioles in the lungs. After time, it induces cardiac vasoconstriction.

Caffeine has generally been used to restore and enhance mental alertness, focus, motivation, and to reduce fatigue. Caffeine increases skeletal muscle performance including strength, power and endurance. Caffeine stimulates all levels of the nervous system and increases metabolism. Caffeine has been shown to increase endurance by upwards of 15% by increasing the utilization of fat stores in the body during exercise.

Caffeine is also commonly used to enhance weight loss. Increased weight loss due to the administration of caffeine may be due, at least in part, by increased lipolysis as fat is metabolized. Caffeine may also enhance weight loss by increasing the basal metabolic rate in humans which increases energy expenditure. (Yoshida et al., Relationship between basal metabolic rate, thermogenic response to caffeine, and body weight loss following low calorie and exercise treatment in obese women. Int. J. Obes Relat Metab Disord. 1994. 18(5): 345-50)

Caffeine can have a synergistic effect when administered with other thermogenic compounds.

### Anti-Inflammatory Compounds

Inflammation is implicated in many diseases including diabetes and heart disease which are closely connected with obesity. Inflammation makes cells less responsive to the hormone leptin which is associated with increasing metabolism and decreasing appetite. Systemic inflammation can lead to leptin resistance which makes it harder to lose weight. In light of the association between systemic inflammation and the difficulty in losing weight, the inventors sought to discover if the addition of an anti-inflammatory ingredient to a weight loss composition would result in increased weight loss.

The anti-inflammatory ingredient can be any compound that is capable of inducing an anti-inflammatory effect when administered to a subject. Examples of anti-inflammatory ingredients includes, but is not limited to: white willow bark extract; acetylsalicylic acid; Harpagophytum (e.g. Devil's claw); hyssop; ginger; tumeric; bromelain; Arnica montana; salicylic acid; and compounds, mixtures or extracts thereof.

### White Willow Bark

White Willow Bark Extract is obtained from the white willow tree, Salix Alba. It has generally been used as an anti-inflammatory and fever reducer and is effective in relieving headaches. White Willow Bark Extract functions by lowering the body's levels of prostaglandins which are responsible for causing inflammation and pain. Recent studies have shown that White Willow Bark Extract possesses antioxidant and immune-enhancing properties. White Willow Bark contains salicin which is a component of aspirin. In fact, White Willow Bark is commonly known as “herbal aspirin”.

White Willow Bark Extract can be used as an appetite depressant since it can lower inflammation thus allowing levels of leptin to increase. White Willow Bark can work synergistically with caffeine to promote thermogenesis.

### Cortisol Level Reducing Compounds

Chronic stress can result in chronically high cortisol levels. Elevated cortisol levels are associated with difficulty in regulating blood glucose levels which may lead to diabetes and insulin resistance. In addition high cortisol levels are associated with high blood pressure, increased stomach acid, decreased immune function, inflammation, and decreased bone formation. High cortisol levels are also associated with weight gain and increased fat storage.

The cortisol level reducing ingredient can be any compound that is capable of lowering cortisol levels in a subject when administered. Examples of ingredients that lower cortisol levels include, but are not limited to: phosphatidylserine; black tea extract; phosphatidylcholine; phosphatidylethanolamine; phosphatidylinositol; rhodiola (e.g. rhodiola rosea); ginseng (e.g. eleuthero senticoccus); magnesium orotate; magnolia bark extract; vitamin C; and compounds and mixtures thereof.

### Phophatidylserine

Phosphatidylserine (PS) is a phospholipid having amphiphilic properties. PS is located in the internal layer of the cell membrane and helps to maintain proper membrane fluidity which impacts most membrane functions. PS also modulates the activity of receptors, ion channels, enzymes and signaling molecules. PS is most concentrated in organs with high metabolic activity such as the brain, heart, lungs, liver and skeletal muscle. It can function to inhibit protein catabolism and promote health and fitness in humans. It speeds up muscle recovery, prevents muscle soreness, and improves well-being. (Starks, M. A. et al., The effects of phosphatidylserine on endocrine response to moderate intensity exercise. J Int Soc Sports Nutr. 2008; 5:11) Phosphatidylserine has been known to increase the stamina and endurance of athletes and reduce muscle soreness. Studies linking PS supplementation to increased stamina and endurance generally prescribe doses of 750 mg and above.

The earliest forms of phosphatidylserine were derived from the brains of cows. However, given the possible health risk involved with relation to Mad Cow Disease, phosphatidylserine is now usually derived from soybeans.

PS can suppress the release of excess cortisol that results from the physical stress of exercise or dieting and has been found to be highly effective at lowering cortisol levels by blunting the hypothalamus reaction to stress. This suppression allows available ingested protein to be utilized for developing muscle as well as alleviating the antagonistic effect of cortisol on amino acid entry into muscle for the synthesis of new muscle growth. It increases the rate of glucose transported into muscle cells and guards against the loss of nutrients from those cells.

Phosphatidylserine (PS) has been shown to effectively reduce cortisol levels that result from physical or mental stress on the body. Since elevated cortisol levels are associated with weight gain, the inventors explored whether phosphatidylserine, which lowers cortisol levels, could be used to effectuate weight loss. PS has not previously been used to promote weight loss. The inventors discovered a synergistic effect in the combination of a thermogenic, an anti-inflammatory, and a cortisol level reducer in effectuating weight loss in a subject. Specifically the combination of caffeine, white willow bark extract, and phosphatidylserine resulted in a synergistic combination that increased weight loss as compared to controls.

The weight loss composition can contain additional inactive ingredients including, but not limited to, dicalcium phosphate, steric acid, magnesium stearate, croscarmellose, micro-crystalline cellulose, and silicon dioxide.

The weight loss composition of the present invention can be administered in a variety of ways to a subject. The easiest route of administration is orally. Using this administration method, the active and inactive ingredients can be combined with a pharmaceutically acceptable carrier in a tablet, capsule or liquid form. A tablet or capsule may be the easiest mode of administration since these forms would allow for easy portability of the composition.

Each tablet/capsule may contain from between about 1 mg and about 400 mg of at least one thermogenic ingredient. A beneficial amount of the at least one thermogenic ingredient in each tablet/capsule is about 200 mg. The amount of the at least one thermogenic ingredient in the weight loss composition consumed daily can be between about 1 mg and about 1200 mg. A beneficial amount of the at least one thermogenic ingredient consumed per day is about 600 mg. In one beneficial embodiment, the at least one thermogenic ingredient can be caffeine administered in 3 doses with each tablet/capsule containing about 200 mg of caffeine.

Each tablet/capsule may contain from between about 1 mg to about 550 mg of at least one anti-inflammatory ingredient. A beneficial amount of the at least one anti-inflammatory ingredient in each tablet/capsule is about 550 mg. The amount of the at least one anti-inflammatory ingredient in the weight loss composition consumed daily can be between about 1 mg and about 1650 mg. A beneficial amount of the at least one anti-inflammatory ingredient consumed per day is about 1650 mg. In one beneficial embodiment, the at least one anti-inflammatory ingredient can be white willow bark extract administered in 3 doses with each tablet/capsule containing about 550 mg of white willow bark extract.

Each tablet/capsule may contain from between about 1 mg to about 400 mg of at least one cortisol level reducing ingredient. A beneficial amount of the at least one cortisol level reducing ingredient in each tablet/capsule is about 200 mg. The amount of the at least one cortisol level reducing ingredient in the weight loss composition consumed daily can be between about 1 mg and about 1200 mg. A beneficial amount of the at least one cortisol level reducing ingredient consumed per day is about 600 mg. In one beneficial embodiment, the at least one cortisol level reducing ingredient can be phosphatidylserine administered in 3 doses with each tablet/capsule containing about 200 mg of phosphatidylserine.

The weight loss composition can be administered orally such as in a tablet or capsule. Each capsule can be taken with about 8 oz. of water. In this form, the dosage can be 1 tablet/capsule administered about 3 times per day about 30 minutes prior to each meal. The dosage can be administered for 5 days with 2 days “off” in which no composition is administered. Alternatively, the composition can be administered for 7 days a week with 1 capsule administered about 2 times per day about 30 minutes prior to a meal. The composition can be administered in a continuous fashion or can alternatively be administered in a cycle in which the composition is taken for a certain amount of time followed a certain amount of time where the composition is not taken. Some non-limiting examples of a typical cycle include: being “on” (administering the composition) for about 6 weeks and being “off” (no administration of the composition) for 2 weeks before beginning a new cycle; being “on” for about 4 weeks and “off” for about 1 week; being “on” for about 8 weeks and being “off” for about 2 weeks. Any combination of weeks/days “on” alternated with a shorter period “off” can be readily determined by one of ordinary skill in the art.

The composition may be taken in combination with a healthy diet and exercise in order to maximize results.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A composition for weight loss in a human comprising:
an effective amount of at least one thermogenic ingredient;
an effective amount of at least one anti-inflammatory ingredient; and
an effective amount of at least one cortisol level reducing ingredient.

2. The composition of claim 1, wherein the at least one thermogenic ingredient is caffeine.

3. The composition of claim 2, wherein the at least one anti-inflammatory ingredient is white willow bark extract.

4. The composition of claim 3, wherein the at least one cortisol level reducing ingredient is phosphatidylserine.

5. The composition of claim 2, wherein the amount of caffeine in the composition is between about 1 mg and about 1200 mg.

6. The composition of claim 3, wherein the amount of white willow bark extract in the composition is between about 1 mg and about 1650 mg.

7. The composition of claim 4, wherein the amount of phosphatidylserine in the composition is between about 1 mg and about 1200 mg.

8. A composition for weight loss in a human comprising:
an effective amount of caffeine;
an effective amount of white willow bark extract; and
an effective amount of phosphatidylserine.

9. The composition of claim 8, wherein the amount of caffeine in the composition is about 200 mg per dose.

10. The composition of claim 8, wherein the amount of white willow bark extract in the composition is about 550 mg per dose.

11. The composition of claim 8, wherein the amount of phosphatidylserine in the composition is about 200 mg per dose.

12. The composition of claim 8, wherein each dose is administered orally.

13. The composition of claim 8, wherein one dose is administered about 3 times per day.

14. The composition of claim 13, wherein the composition is administered for 5 consecutive days per week.

15. The composition of claim 8, wherein each dose is administered about 30 minutes prior to a meal.

16. A method of promoting weight loss in a human comprising administering a weight loss composition comprising:
an effective amount of caffeine;
an effective amount of white willow bark extract; and
an effective amount of phosphatidylserine.

17. The method of claim 16, wherein the composition is administered orally.

18. The method of claim 16, wherein the composition is administered about 3 times per day.

19. The method of claim 18, wherein the composition is administered for 5 consecutive days per week.

20. The method of claim 16, wherein the composition is administered about 30 minutes prior to a meal.
